(19) United States
(12) Patent Application Publication
(10) Pub. No.: US 2008/0213385 A1
(43) Pub. Date: Sep. 4, 2008

(54) Formulations for 7- (T-Butoxy) Iminomethyl Camptothecin

(76) Inventors: Oskar Kalb, Lorrach (DE); Isabel Ottinger, Freiburg (DE); Walter Stebler, Liestal (CH); Agnes Taillardat, Seewen SO (CH); Wolfgang Wirth, Arisdorf (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/996,638

(22) PCT Filed: Aug. 8, 2006
(86) PCT No.: PCT/EP2006/065159

§ 371 Date: Jan. 24, 2008

Related U.S. Application Data
(60) Provisional application No. 60/707,190, filed on Aug. 10, 2005.

(51) Int. Cl. A61K 31/4375; A61K 9/14; A61P 35/00

(52) U.S. Cl. 424/499; 514/283; 424/489; 424/501

(57) ABSTRACT

The present invention relates to nanoparticulate compositions in which the active agent is a topoisomerase I inhibitor and pharmaceutical compositions comprising the nanoparticulate compositions that are useful for the treatment and prevention of proliferative diseases including cancer.

### FIELD OF THE INVENTION

The present invention relates to nanoparticulate compositions in which the active agent is a topoisomerase I inhibitor and pharmaceutical compositions comprising the nanoparticulate compositions that are useful for the treatment and prevention of proliferative diseases including cancer.

### BACKGROUND OF THE INVENTION

Camptothecin derivatives are a class of compounds described in U.S. Pat. No. 6,242,457 and present highly specific difficulties in relation to administration generally and galenic compositions, in particular, including in particular problems of drug bioavailability because these derivatives have very poor solubility.

Nanoparticulate compositions are particles consisting of a poorly soluble therapeutic agent having adsorbed onto the surface thereof a surface stabilizer. Methods of making nanoparticulate compositions are described, for example, in U.S. Pat. Nos. 5,518,187 and 5,862,999, both for “Method of Grinding Pharmaceutical Substances”; U.S. Pat. No. 5,718,388, for “Continuous Method of Grinding Pharmaceutical Substances”; and U.S. Pat. No. 5,510,118 for “Process of Preparing Therapeutic Compositions Containing Nanoparticles”.

### SUMMARY OF THE INVENTION

The present invention relates to nanoparticulate compositions comprising a topoisomerase I inhibitor, in particular, 7-t-butoxyiminomethylcamptothecin, as the active agent, and at least one surface stabilizer.

The present invention also relates to a method of making the nanoparticulate compositions of the present invention. Such a method comprises contacting particles of 7-t-butoxyiminomethylcamptothecin and at least one surface stabilizer for a time and under conditions sufficient to provide a nanoparticulate composition. The one or more surface stabilizers can be contacted with 7-t-butoxyiminomethylcamptothecin either before, during, or after size reduction of 7-t-butoxyiminomethylcamptothecin.

The present invention also relates to pharmaceutical compositions comprising the nanoparticulate compositions of the present invention and a pharmaceutically acceptable carrier, as well as any pharmaceutical acceptable excipients.

The present invention also relates to methods of treatment using the pharmaceutical compositions of the present invention for conditions, such as proliferative diseases or diseases that are associated with or triggered by persistent angiogenesis.

### DETAILED DESCRIPTION OF THE DRAWINGS

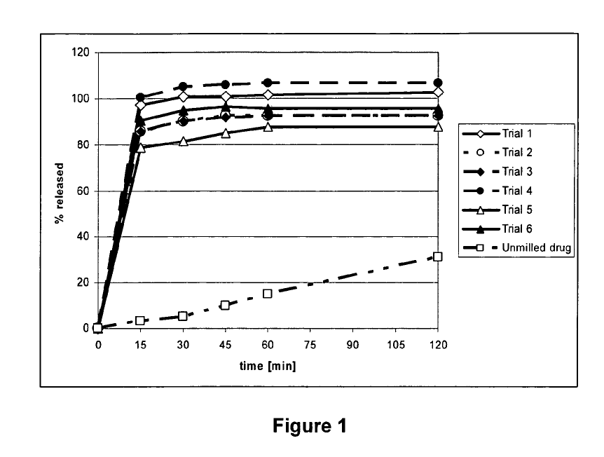

FIG. 1 illustrates in vitro dissolution rate profiles of nano-suspensions and the pure drug substance as described in Example 1. Legend: nano-suspensions of trial 1 to 6 and unmilled drug.

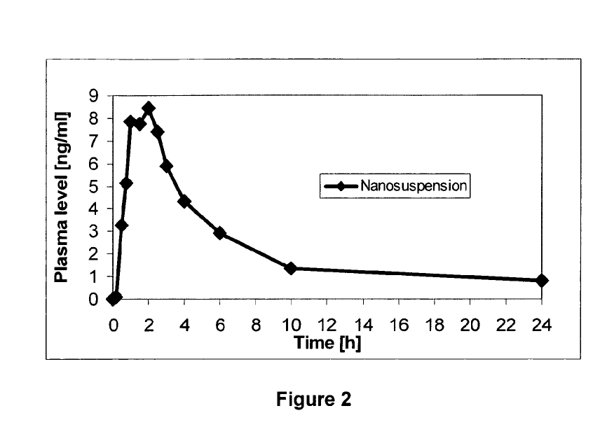

FIG. 2 illustrates in vivo dog bioavailability from the nano-suspension as described in Example 2

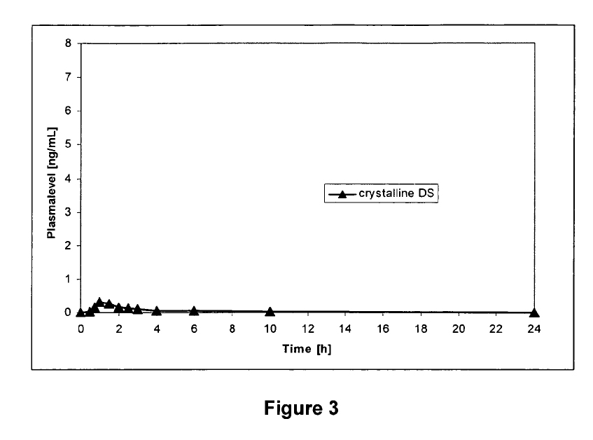

FIG. 3 illustrates in vivo dog bioavailability from the pure drug substance as described in Example 2.

### DETAILED DESCRIPTION OF THE INVENTION

The nanoparticulate compositions of the present invention comprise of 7-t-butoxyiminomethylcamptothecin having an effective average particle size of less than about 4 microns and preferably at least one surface stabilizer.

An additional feature of the nanoparticulate compositions of the present invention is that the compositions redisperse such that the effective average particle size of the redispersed 7-t-butoxyiminomethylcamptothecin particles are less than about 2-4 microns. This is significant, as if upon administration the nanoparticulate 7-t-butoxyiminomethylcamptothecin particles present in the compositions of the invention did not redisperse to a substantially small particle size, then the dosage form may lose the benefits afforded by formulating 7-t-butoxyiminomethylcamptothecin into a nanoparticulate particle size.

Preferably, the re-dispersed particles of the invention have an effective average particle size, by weight distribution, of less than about 4,000 nm, preferably less than 2,000 nm, more preferably less than about 1,000 nm, and most preferably less than about 500 nm as measured by light-scattering methods, microscopy or other appropriate methods.

“Active agent”, as used herein, includes 7-t-butoxyiminomethylcamptothecin having the following structure known as Compound A:

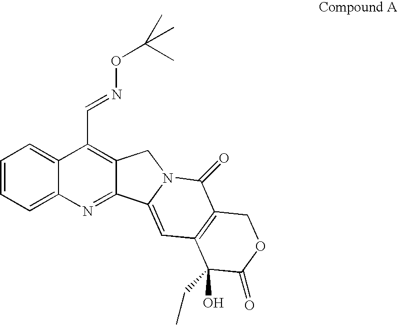

The preferred active agent can be in free or pharmaceutically acceptable salt form, in the form of their possible enantiomers, diastereoisomers and relative mixtures, polymorphs, amorphous, partially amorphous forms, solvates, their active metabolites and prodrugs.

In accordance with the present invention the active agent may be present in an amount by weight from about 0.001% to about 30% by weight of the composition of the invention. The active agent is preferably present in an amount of about 0.01% to about 5% by weight of the composition.

“Poorly water soluble”, as used herein, means having a solubility in water at 20° C. of less than 1%, e.g., 0.01% weigh/volume, i.e., a “sparingly soluble to very slightly soluble drug” as described in Remington: The Science and Practice of Pharmacy, 19^{th} Edition, A. R. Gennaro, Ed., Mack Publishing Company, US, Vol. 1, p. 195 (1995).

By “an effective average particle size of less than about 4,000 nm” it is meant that at least 50% of the nanoparticulate active agent particles have a particle size of less than about 4,000 nm, by weight, when measured by the below-noted techniques. Preferably, at least about 70%, about 90%, about 95% or about 99% of the nanoparticulate active agent particles have a particle size of less than the effective average, i.e., less than about 4,000 mm, less than about 3,000 nm, less than about 2,000 nm, etc. As used herein, particle size is determined on the basis of the weight average particle size as measured by conventional particle size measuring techniques well-known to those skilled in the art. Such techniques include, e.g., sedimentation field flow fractionation, photon correlation spectroscopy, light scattering and disk centrifugation.

The terms “effective amount” or “pharmaceutically effective amount” of a nanoparticle formulation, as provided herein, refer to a nontoxic but sufficient amount of the nanoparticle formulation to provide the desired response, and corresponding therapeutic effect, in an amount sufficient to effect treatment of the subject, as defined below. As will be pointed out below, the exact amount required will vary from subject to subject, depending on the species, age, and general condition of the subject, the severity of the condition being treated, mode of administration and the like. An appropriate “effective” amount in any individual case may be determined by one of ordinary skill in the art using routine experimentation.

The phrase “pharmaceutically acceptable” or “pharmacologically acceptable” means a material which is not biologically or otherwise undesirable, i.e., the material may be administered to an individual along with the nanoparticle formulation without causing any undesirable biological effects or interacting in a deleterious manner with any of the components of the composition in which it is contained.

### I. Surface Stabilizer

Combinations of more than one surface stabilizer can be used in the invention. Preferred primary surface stabilizers include, but are not limited to, hydroxypropyl methylcellulose, hydroxypropylcellulose, polyvinylpyrrolidone, random copolymers of vinyl pyrrolidone and vinyl acetate or a combination thereof. Preferred secondary surface stabilizers include, but are not limited to, poloxamers, sodium lauryl sulfate and dioctylsulfosuccinate.

Other surface stabilizers which can be employed in the invention include, but are not limited to, known organic and inorganic pharmaceutical excipients. Such excipients include various polymers, low molecular weight oligomers, natural products and surfactants. Surface stabilizers include nonionic, cationic, ionic and zwitterionic surfactants.

Representative examples of surface stabilizers include gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers, such as cetomacrogol 1000), polyoxyethylene castor oil derivatives, polyoxyethylene sorbitan fatty acid esters (e.g., the commercially-available Tweens®, such as, e.g., Tween 20® and Tween® (ICI Specialty Chemicals)); polyethylene glycols (e.g., Carbowaxs 3550® and 934® (Union Carbide)), polyoxyethylene stearates, colloidal silicon dioxide, phosphates, carboxymethylcellu lose calcium, carboxymethylcellu lose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione and triton), poloxamers (e.g., Pluronics F68® and F108®, which are block copolymers of ethylene oxide and propylene oxide); poloxamines (e.g., Tetronic 9080, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 15080 (T-1508) (BASF Wyandotte Corporation), Tritons X-2000, which is an alkyl aryl polyether sulfonate (Rohm and Haas); Crodestas F-100®, which is a mixture of sucrose stearate and sucrose distearate (Croda Inc.); p-isononylphenoxypoly-(glycidol), also known as Olin-10G® or Surfactant 10-Go (Olin Chemicals, Stamford, Conn.); Crodestas SL-400 (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl-β-D-glucopyranoside; n-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-noyl-β-D-glucopyranoside; octanoyl-N-methylglucamide; n-octyl-β-D-glucopyranoside; octyl-β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Such exemplary cationic surface stabilizers and other useful cationic surface stabilizers are described in J. Cross and E. Singer, Cationic Surfactants: Analytical and Biological Evaluation, Marcel Dekker (1994); P. and D. Rubingh, Ed., Cationic Surfactants: Physical Chemistry, Marcel Dekker (1991); and J. Richmond, Cationic Surfactants: Organic Chemistry, Marcel Dekker (1990).

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference. The surface stabilizers are commercially-available and/or can be prepared by techniques known in the art.

The concentration of the at least one surface stabilizer can vary from about 0.5% to about 99.999%, from about 5.0% to about 99.9%, or from about 10% to about 99.5%, by weight, based on the total combined dry weight of the active agent and at least one surface stabilizer, not including other excipients.

If a combination of two or more surface stabilizers is employed in the composition, the concentration of at least one primary surface stabilizer can vary from about 0.01% to about 99.5%, from about 0.1% to about 95%, or from about 0.5% to about 90%, by weight, based on the total combined dry weight of the active agent not including other excipients.

### II. Processes for Preparing the Nanoparticle Compositions

The nanoparticulate compositions of the present invention can be made using, e.g., milling, homogenization or precipitation techniques.

Milling the active agent to obtain a nanoparticulate dispersion comprises dispersing particles of the active agent in a liquid dispersion medium in which the active agent is poorly soluble, followed by applying mechanical means in the presence of grinding media to reduce the particle size of the active agent to the desired effective average particle size. The dispersion medium can be, e.g., water, ethanol, t-butanol, glycerin, polyethylene glycol (PEG), hexane or glycol.

In one embodiment, aqueous nanomilling of the active agent is conducted in the presence of hydrophilic stabilizer.

The active agent particles can be reduced in size in the presence of at least one surface stabilizer. Alternatively, the active agent particles can be contacted with one or more surface stabilizers after attrition. Other compounds, such as a diluent, can be added to the active agent/surface stabilizer composition either before, during or after the size reduction process. Dispersions can be manufactured continuously or in a batch mode.

In another embodiment, the nanoparticulate composition is prepared by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, e.g.,
(1) dissolving the active agent in a suitable solvent;
(2) adding the formulation from step (1) to a solution comprising at least one surface stabilizer; and
(3) precipitating the formulation from step (2) using an appropriate non-solvent.
   The method can be followed by removal of any formed salt, if present, by dialysis or diafiltration and concentration of the dispersion by conventional means.

In another embodiment, the nanoparticle compositions are prepared by homogenization methods. Such a method comprises dispersing the active agent particles in a liquid dispersion medium, followed by subjecting the dispersion to homogenization to reduce the particle size of the active agent to the desired effective average particle size. The active agent particles can be reduced in size in the presence of at least one surface stabilizer. Alternatively, the active agent particles can be contacted with one or more surface stabilizers either before or after attrition. Other compounds, such as a diluent, can be added to the active agent/surface stabilizer composition either before, during or after the size reduction process. Dispersions can be manufactured continuously or in a batch mode.

### III. Pharmaceutical Compositions and Methods of Treatment

The pharmaceutical compositions of the present invention also include one or more physiologically acceptable carriers, adjuvants or vehicles, collectively referred to as carriers. The compositions can be formulated for oral administration in solid, or liquid form, and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents and other excipients. Such excipients are known in the art. Examples of filling agents are lactose monohydrate, lactose anhydrous, microcrystalline cellulose, such as Avicel® PH101 and Avicel® PH102, microcrystalline cellulose and silicified microcrystalline cellulose (ProSolv SMCC®), and various starches; examples of binding agents are various celluloses and cross-linked polyvinylpyrrolidone. Suitable lubricants, including agents that act on the flowability of the powder to be compressed, are colloidal silicon dioxide, such as Aerosil® 200, talc, stearic acid, magnesium stearate, calcium stearate and silica gel. Examples of sweeteners are any natural or artificial sweetener, such as sucrose, xylitol, sodium saccharin, cyclamate, aspartame, sucralose, maltitol and acsulfame. Examples of flavoring agents are Magnasweet® (trademark of MAFCO), bubble gum flavor, and fruit flavors, and the like. Examples of preservatives are potassium sorbate, methylparaben, propylparaben, benzoic acid and its salts, other esters of parahydroxybenzoic acid, such as butylparaben; alcohols, such as ethyl or benzyl alcohol. Suitable diluents include pharmaceutically acceptable inert fillers, such as microcrystalline cellulose, lactose, dibasic calcium phosphate, saccharides and/or mixtures of any of the foregoing. Examples of diluents include microcrystalline cellulose, such as Avicel® PH101 and Avicel® PH1 02; lactose, such as lactose monohydrate, lactose anhydrous, and Pharmatose® DCL21; dibasic calcium phosphate, such as Emcompress®; mannitol; starch; sorbitol; sucrose; and glucose. Suitable disintegrants include lightly crosslinked polyvinyl pyrrolidone, corn starch, potato starch, maize starch, and modified starches, croscarmellose sodium, cross-povidone, sodium starch glycolate and mixtures thereof. Examples of effervescent agents are effervescent couples, such as an organic acid and a carbonate or bicarbonate. Suitable organic acids include, e.g., citric, tartaric, malic, fumaric, adipic, succinic and alginic acids and anhydrides and acid salts. Suitable carbonates and bicarbonates include, e.g., sodium carbonate, sodium bicarbonate, potassium carbonate, potassium bicarbonate, magnesium carbonate, sodium glycine carbonate, L-lysine carbonate and arginine carbonate. Alternatively, only the sodium bicarbonate component of the effervescent couple may be present.

The nanoparticulate compositions of the invention can be administered to a subject via any conventional means including orally and parenterally. As used herein, the term “subject” is used to mean an animal, preferably a mammal, including a human or non-human. The terms patient and subject may be used interchangeably.

Solid dosage forms for oral administration include, but are not limited to, capsules, tablets, pills, powders and granules. In such solid dosage forms, the active agent is admixed with at least one of the following:
(a) one or more inert excipients (or carriers), such as sodium citrate or dicalcium phosphate;
(b) fillers or extenders, such as starches, lactose, sucrose, glucose, mannitol and silicic acid;
(c) binders, such as carboxymethylcellulose, alignates, gelatin, polyvinylpyrrolidone, sucrose and acacia;
(d) humectants, such as glycerol;
(e) disintegrating agents, such as cross-linked starches, polyvinylpyrrolidone XL, agar-agar, calcium carbonate, potato or tapioca starch, alginic acid, certain complex silicates and sodium carbonate;
(f) solution retarders, such as paraffin;
(g) absorption accelerators, such as quaternary ammonium compounds;
(h) wetting agents, such as cetyl alcohol and glycerol monostearate;
(i) adsorbents, such as kaolin and bentonite; and
(j) lubricants, such as talc, calcium stearate, magnesium stearate, solid polyethylene glycols, sodium lauryl sulfate or mixtures thereof.
   For capsules, tablets and pills, the dosage forms may also comprise buffering agents.

Liquid nanoparticulate dosage forms for oral administration include pharmaceutically acceptable emulsions, solutions, suspensions, syrups and elixirs. In addition to the active agent, the liquid dosage forms may comprise inert diluents commonly used in the art, such as water or other solvents, co-solvents, solubilizing agents and emulsifiers. Non-limiting examples of solvents and co-solvents include ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol and dimethyl isosorbide, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring and perfuming agents.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex and diet of the patient; the time of administration, route of administration and rate of excretion of the agent; the duration of the treatment; drugs used in combination or coincidental with the specific agent; and like factors well-known in the medical arts.

The pharmaceutical compositions of the present invention are useful for treating proliferative diseases or diseases that are associated with or triggered by persistent angiogenesis. A proliferative disease is mainly a tumor disease (or cancer) (and/or any metastases). The inventive compositions are particularly useful for treating a tumor which is a breast cancer, lung cancer, gastrointestinal cancer, including esophageal, gastric, small bowel, large bowel and rectal cancer, glioma, sarcoma, such as those involving bone, cartilage, soft tissue, muscle, blood and lymph vessels, ovarian cancer, myeloma, female cervical cancer, endometrial cancer, head and neck cancer, mesothelioma, renal cancer, ureter, bladder and urethral cancers, prostate cancer, skin cancers and melanoma. In particular, the inventive compositions are particularly useful for treating:

(i) a breast tumor; a lung tumor, e.g., non-small cell lung tumor; a gastrointestinal tumor, e.g., a colorectal tumor; or a genitourinary tumor, e.g., a prostate tumor;

(ii) a proliferative disease that is refractory to the treatment with other chemotherapeutics; or

(iii) a tumor that is refractory to treatment with other chemotherapeutics due to multidrug resistance.

In a broader sense of the invention, a proliferative disease may furthermore be a hyperproliferative condition, such as a leukemia, lymphoma, multiple myeloma.

The following examples are given to illustrate the present invention. It should be understood, however, that the invention is not to be limited to the specific conditions or details described in these examples.

### EXAMPLE 1

Table 1 shows the composition of the aqueous suspensions subjected to nano-milling.

TABLE 1
Aqueous Nano-milling: Composition of the Aqueous Suspensions
Prior Milling

| Trial | Drug [%]* | Stabilizer | Stabilizer [%]* | Milling time |
|---|---|---|---|---|
| 1 | 1 | Povidone K-30 | 0.2 | 7 hours |
| 2 | 1 | HPMC 3 cps | 0.2 | 3.5 hours |
| 3 | 1 | HPMC 3 cps | 0.2 | 7 hours |
| 4 | 1 | HPMC 3 cps | 0.2 | 24 hours |
| 5 | 1 | HPC, low viscosity | 0.2 | 7 hours |
| 6 | 1 | Poloxamer 188 | 0.2 | 7 hours |

*% (w/w) in aqueous suspension

The aqueous nano-milling was performed in a ball mill using yittrium dropped zirconia beads (0.5-0.6 mm in 0). For all trials the batch size was approximately 70 g. Prior to milling the beads were conditioned with 1% stabilizer solution for 24 hours at 1,200 rpm (minimal speed, 80 mL solution for 160 mL beads), rinsed with demineralized water until conductivity reading was the same as that of the water, placed in a 150-200° C. oven until dry and cooled to room temperature before use. Milling was performed at 3,200 rpm and milling times as outlined in Table 1 were used. Before and after milling the aqueous suspensions were characterized with respect to particle size distribution, appearance (microscopic pictures and laser light scattering), and dissolution rate and assay/degradation.

The dissolution rate testing was done using an USP2 apparatus, paddle, 50 rpm, 37° C., 0.3% SDS in 1,000 mL water, n=3. For dissolution rate testing, the aqueous suspensions were diluted 1:5 with water and filled into vials (0.5 mg drug substance/vial).

The results of the in vitro characterization, including dissolution rate testing are given in Table 2.

TABLE 2
Results of the In Vitro Characterization of the Aqueous Nanosuspensions

| | | | | Dissolution rate: % | | | |
|---|---|---|---|---|---|---|---|
| | PSD (light | PSD, | | released, mean (n = 3), | | | |
| | scattering, | appearance light microscopy | | after | | | |
| Trial | after milling) | Prior milling | After milling | 15 min | 30 min | 60 min | 120 min |
| 1 | x10 = 0.2 μm | Column shaped | Fine particles | 97 | 101 | 102 | 103 |
| | x50 = 0.5 μm | particles, length | (non visible) | | | | |
| | x90 = 1.9 μm | <5 - app. 100 μm | | | | | |
| 2 | x10 = 0.3 μm | Column shaped | Fine particles | 86 | 90 | 93 | 93 |
| | x50 = 0.8 μm | particles, length | (non visible) | | | | |
| | x90 = 2.6 μm | <5 - app. 100 μm | | | | | |
| 3 | x10 = 0.2 μm | Column shaped | Fine particles | 86 | 90 | 92 | 92 |
| | x50 = 0.9 μm | particles, length | (non visible) | | | | |
| | x90 = 13.7 μm | <5 - app. 100 μm | | | | | |
| 4 | x10 = 0.2 μm | Column shaped | Fine particles | 100 | 105 | 107 | 107 |
| | x50 = 3.7 μm | particles, length | (non visible) | | | | |
| | x90 = 27.4 μm | <5 - app. 100 μm | | | | | |
| 5 | x10 = 9.0 μm | Column shaped | Fine particles | 79 | 81 | 88 | 88 |
| | x50 = 37.7 μm | particles, length | (non visible) | | | | |
| | x90 = 92.4 μm | <5 - app. 30 μm | | | | | |
| 6 | x10 = 0.4 μm | Column shaped | Fine particles | 91 | 95 | 96 | 96 |
| | x50 = 1.5 μm | particles, length | (non visible) | | | | |
| | x90 = 6.1 μm | <5 - app. 100 μm | | | | | |
| Unmilled | x10 = 2 μm | Not determined | Not applicable | 3 | 5 | 15 | 31 |
| drug | x50 = 9 μm | | | | | | |
| | x90 = 35 μm | | | | | | |

PSD = particle size distribution

As indicated in Table 2 all aqueous nano-suspensions show very high dissolution rates compared to the unmilled drug substance. Almost 100% drug substance was released within 15 minutes. No significant difference was observed between the variants.

The analysis by light microscope revealed that the particle size of the active agent in the suspensions was significantly reduced by milling. Particle size changed from up to approximately 100 μm to particles which were not visible anymore. The particle size distribution measured by laser light scattering indicates that particles with ×90 <3 μm were obtained for variant 1 and 2. Slightly larger particles were observed for 3 and 6, while aggregation formation was seen for variant 5 containing Poloxamer 188 as stabilizer.

Macroscopically, the milled suspensions are yellowish and opaque. The same appearance is observed after 1:5 dilution of the suspensions with water.

The results summarized above indicate that aqueous nanomilling is feasible for the active agent. Significant reduction in particle size could be achieved by milling. All variants showed improved dissolution performance (nearly 100% release in 15 minutes) compared to the non-milled drug substance.

### EXAMPLE 2

The bioavailability of 7-t-butoxyiminomethylcamptothecin is compared as it is determinable after administration of unmilled drug substance in a dry powder formulation (hard capsule) and of a composition according to the present invention (liquid form).

Administered form: 0.5 mg 7-t-butoxyiminomethylcamptothecin per dog.

The composition according to the present invention corresponds to trial 2 from Example 1.

### Method

Six (6) dogs completed the study. Each of the dog received both formulations. Blood samples for the determination of 7-t-butoxyiminomethylcamptothecin in plasma were taken before dosing, and then 10 minutes, 30 minutes, 45 minutes, 1 hour, 1.5 hours, 2 hours, 2.5 hours, 3 hours, 4 hours, 6 hours, 10 hours and 24 hours after drug intake. The individual concentrations of 7-t-butoxyiminomethylcamptothecin in heparinized plasma were determined for each sample by a liquid chromatography/tandem mass spectroscopy in positive electrospray ionization mode (positive ESI-LC/MS-MS). Heparinized plasma samples were prepared for analysis by liquid-liquid extraction and evaporation of the supernatant to dryness before reconstitution in the injection medium. The limit of quantification was 0.1 ng/mL.

Results (see also FIGS. 2 and 3)

| | Formul. | |
|---|---|---|
| | Example 1 | Formul. Pure Drug |
| | Trial 2 | Substance in Capsule |
| PK parameters | Mean (CV %) | Mean (CV %) |
| Actual dose [mg/kg] | 0.0369 (5) | 0.0443 (15) |
| AUC(0-24 h) [(ng/mL) · h] | 55.0 (41) | 0.86 (155) |
| AUC(0-24 h)/dose, [(ng/mL) · | 1489 (40) | |
| h/(mg/kg)] | | |
| Median | 1343 | |
| AUC(0-∞) [(ng/mL) · h] | 83.4 (56) | |
| AUC(0-∞)/dose [(ng/mL) · | 1489 (40) | |
| h/(mg/kg)] | | |
| Median | 1343 | |
| Cmax [ng/mL] | 11.0 (28) | 0.37 (70) |
| Cmax/dose [(ng/mL)/(mg/kg)] | 296 (25) | 8.2 (68) |
| Median | 285 | |
| tmax [h] | 1.50 (52) | 1.2 (27) |
| tmax [h] range | 1 to 2.5 | |
| t½ [h] | 7 to 18 | |

1. A composition comprising:
(a) nanoparticles of 7-t-butoxyiminomethylcamptothecin;
(b) at least one surface stabilizer, wherein the nanoparticles have an effective average particle size of less than about 4,000 nm; and
(c) showing at least a 1.5-fold better bioavailability than the unformulated drug in a subject.

2. The composition of claim 1, wherein the 7-t-butoxyiminomethylcamptothecin is in free or pharmaceutically acceptable salt form, in the form of their possible enantiomers, diastereoisomers and relative mixtures, polymorphs, amorphous, partially amorphous forms, solvates, their active metabolites and prodrugs any combination thereof.

3. The composition of claim 1, wherein the composition shows at least a 1.5-fold better bioavailability when compared with 7-t-butoxyiminomethylcamptothecin in free form.

4. The composition of claim 1, wherein the effective average particle size of the nanoparticulate particles is selected from the group consisting of less than about 3,000 nm, less than about 2,000 nm, less than about 1,000 nm, less than about 500 nm.

5. The composition of claim 1, wherein the composition further comprises one or more pharmaceutically acceptable excipients, surface stabilizers or a combination thereof.

6. The composition of claim 1, wherein 7-t-butoxyiminomethylcamptothecin is present in an amount selected from the group consisting of from about 99.5% to about 0.001%, from about 95% to about 0.1%, and from about 90% to about 0.5%, by weight, based on the total combined weight of the 7-t-butoxyiminomethylcamptothecin and at least one surface stabilizer, not including other excipients.

7. The composition of claim 1, wherein the at least one surface stabilizer is present in an amount selected from the group consisting of from about 0.5% to about 99.999%, from about 5.0% to about 95%, and from about 10% to about 99.5%, by weight, based on the total combined dry weight of 7-t-butoxyiminomethylcamptothecin and at least one surface stabilizer, not including other excipients.

8. The composition of claim 1, wherein the at least one surface stabilizer is selected from hydroxypropyl methylcellulose, hydroxypropylcellulose, polyvinylpyrrolidone, random copolymers of vinyl pyrrolidone and vinyl acetate, sodium lauryl sulfate, dioctylsulfosuccinate, poloxamers, gelatin, casein, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters; polyethylene glycols, polyoxyethylene stearates, colloidal silicon dioxide, phosphates, carboxymethylcellu lose calcium, carboxymethylcellu lose sodium, methylcellulose, hydroxyethylcellulose, hydroxypropylmethylcellulose phthalate, noncrystalline cellulose, magnesium aluminium silicate, triethanolamine, polyvinyl alcohol, 4-(1,1,3,3-tetramethylbutyl)-phenol polymer with ethylene oxide and formaldehyde; poloxamines; Tetronic 15080, alkyl aryl polyether sulfonate; a mixture of sucrose stearate and sucrose distearate; p-isononylphenoxypoly-(glycidol), C₁₈H₃₇CH₂(CON(CH₃)—CH₂(CHOH)₄(CH₂OH)₂; decanoyl-N-methylglucamide; n-decyl-β-D-glucopyranoside; n-decyl-3-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-noyl-β-D-glucopyranoside; octanoyl-N-methylglucamide; n-octyl-β-D-glucopyranoside; octyl-β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate.

9. The composition of claim 1, wherein the composition is in a liquid oral dosage form.

10. The composition of claim 1, wherein the composition is in a solid oral dosage form.

11. The method of claim 1, wherein the formulation is in a parenteral dosage form.

12. A method of making a nanoparticulate composition comprising contacting 7-t-butoxyiminomethylcamptothecin with at least one surface stabilizer for a time and under conditions sufficient to provide a nanoparticulate composition having an effective average particle size of less than about 4,000 nm.

13. The method of claim 12, wherein said contacting comprising grinding.

14. The method of claim 13, wherein said grinding comprising wet grinding.

15. The method of claim 12, wherein said contacting comprises homogenizing.

16. The method of claim 12, wherein said contacting comprises precipitation.

17. The method of claim 12, wherein the effective average particle size of the nanoparticulate particles is selected from the group consisting of less than about 3,000 nm, less than about 2,000 nm, less than about 1,000 nm, less than about 500 nm.

18. The method of claim 12, wherein the at least one surface stabilizer is selected from hydroxypropyl methylcellulose, hydroxypropylcellulose, polyvinylpyrrolidone, random copolymers of vinyl pyrrolidone and vinyl acetate, sodium lauryl sulfate, dioctylsulfosuccinate, poloxamers, gelatin, casein, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters; polyethylene glycols, polyoxyethylene stearates, colloidal silicon dioxide, phosphates, carboxymethylcellu lose calcium, carboxymethylcellu lose sodium, methylcellulose, hydroxyethylcellulose, hydroxypropylmethylcellulose phthalate, noncrystalline cellulose, magnesium aluminium silicate, triethanolamine, polyvinyl alcohol, 4-(1,1,3,3-tetramethylbutyl)-phenol polymer with ethylene oxide and formaldehyde; poloxamines; Tetronic 15080, alkyl aryl polyether sulfonate; a mixture of sucrose stearate and sucrose distearate; p-isononylphenoxypoly-(glycidol), C₁₈H₃₇CH₂(CON(CH₃)—CH₂(CHOH)₄(CH₂OH)₂; decanoyl-N-methylglucamide; n-decyl-β-D-glucopyranoside; n-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-noyl-β-D-glucopyranoside; octanoyl-N-methylglucamide; n-octyl-β-D-glucopyranoside; octyl-β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate.

19. A method of treating a proliferative disease comprising administrating to a patient in need thereof a formulation comprising nanoparticles of 7-t-butoxyiminomethylcamptothecin and at least one surface stabilizer, wherein the nanoparticles have an effective average particle size of less than about 4,000 nm.

20. The method of claim 19, wherein the proliferative disease is breast cancer, lung cancer, gastrointestinal cancer, including esophageal, gastric, small bowel, large bowel and rectal cancer, glioma, sarcoma such as those involving bone, cartilage, soft tissue, muscle, blood and lymph vessels, ovarian cancer, myeloma, female cervical cancer, endometrial cancer, head and neck cancer, mesothelioma, renal cancer, ureter, bladder and urethral cancers, prostate cancer, skin cancers and melanoma. In particular, the inventive compositions are particularly useful for treating:
(i) a breast tumor; a lung tumor, e.g., non-small cell lung tumor; a gastrointestinal tumor, e.g., a colorectal tumor; or a genitourinary tumor, e.g., a prostate tumor;
(ii) a proliferative disease that is refractory to the treatment with other chemotherapeutics; or
(iii) a tumor that is refractory to treatment with other chemotherapeutics due to multidrug resistance.
In a broader sense of the invention, a proliferative disease may furthermore be a hyperproliferative condition, such as a leukemia, lymphoma and multiple myeloma.

21. The method of claim 19, wherein 7-t-butoxyiminomethylcamptothecin is present in an amount selected from the group consisting of from about 99.5% to about 0.001%, from about 95% to about 0.1%, and from about 90% to about 0.5%, by weight, based on the total combined weight of the camptothecin derivative and at least one surface stabilizer, not including other excipients.

22. The method of claim 19, wherein the at least one surface stabilizer is present in an amount selected from the group consisting of from about 0.5% to about 99.999%, from about 5.0% to about 95%, and from about 10% to about 99.5%, by weight, based on the total combined dry weight of 7-t-butoxyiminomethylcamptothecin and at least one surface stabilizer, not including other excipients.

23. The method of claim 19, wherein the at least one surface stabilizer is selected from hydroxypropyl methylcellulose, hydroxypropylcellulose, polyvinylpyrrolidone, random copolymers of vinyl pyrrolidone and vinyl acetate, sodium lauryl sulfate, dioctylsulfosuccinate, poloxamers, gelatin, casein, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters; polyethylene glycols, polyoxyethylene stearates, colloidal silicon dioxide, phosphates, carboxymethylcellu lose calcium, carboxymethylcellu lose sodium, methylcellulose, hydroxyethylcellulose, hydroxypropylmethylcellulose phthalate, noncrystalline cellulose, magnesium aluminium silicate, triethanolamine, polyvinyl alcohol, 4-(1,1,3,3-tetramethylbutyl)-phenol polymer with ethylene oxide and formaldehyde; poloxamines; Tetronic 15080, alkyl aryl polyether sulfonate; a mixture of sucrose stearate and sucrose distearate; p-isononylphenoxypoly-(glycidol), C₁₈H₃₇CH₂(CON(CH₃)—CH₂(CHOH)₄(CH₂OH)₂; decanoyl-N-methylglucamide; n-decyl-β-D-glucopyranoside; n-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-noyl-β-D-glucopyranoside; octanoyl-N-methylglucamide; n-octyl-β-D-glucopyranoside; octyl-β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate.

24. The method of claim 19, wherein the formulation is in a liquid oral dosage form.

25. The method of claim 19, wherein the formulation is in a solid oral dosage form.

26. The method of claim 19, wherein the formulation is in a parenteral dosage form.

27. A dosage form comprising 0.001-100 mg by weight of 7-t-butoxyiminomethylcamptothecin.

28. The dosage form of claim 27, comprising 0.01-25 mg by weight of 7-t-butoxyiminomethylcamptothecin.

29. The dosage form of claim 27, comprising 0.05-10 mg by weight of 7-t-butoxyiminomethylcamptothecin.
